(19) United States
(12) Patent Application Publication
(10) Pub. No.: US 2010/0093062 A1
(43) Pub. Date: Apr. 15, 2010

(54) TRANSFECTION SYSTEM FOR PERKINSUS SPECIES

(75) Inventors: Jose-Antonio Fernandez-Robledo, Baltimore, MD (US); Gerardo Raul Vasta, Columbia, MD (US)

Correspondence Address: INTELLECTUAL PROPERTY / TECHNOLOGY LAW, PO BOX 14329, RESEARCH TRIANGLE PARK, NC, 27709, US

(73) Assignee: UNIVERSITY OF MARYLAND BIOTECHNOLOGY INSTITUTE, Baltimore, MD (US)

(21) Appl. No.: 12/532,335

(22) PCT Filed: Mar. 17, 2008
(86) PCT No.: PCT/US08/57203

§ 371 Date: Dec. 16, 2009

Related U.S. Application Data
(60) Provisional application No. 60/918,921, filed on Mar. 20, 2007.

(51) Int. Cl. C12N 1/11; C12N 15/74

(52) U.S. Cl. 435/258.1; 435/320.1

(57) ABSTRACT

A Perkinsus heterologous expression system that is useful to produce recombinant proteins, e.g., from related parasites of medical and veterinary relevance. This expression system increases the number of genes that can be targeted for protein structure/function studies, production of antigens, and in vitro drug screening. Also described is a transfection system for Perkinsus species, including a transfection vector expressing heterologous DNA encoding one or more Apicomplexa proteins.

### CROSS-REFERENCE TO RELATED APPLICATION

The present application claims the benefit under 35 USC 119 of U.S. Provisional Patent Application No. 60/918,921 filed Mar. 20, 2007 in the names of Jose-Antonio Fernandez-Robledo and Gerardo Raul Vasta for “A transfection system for Perkinsus species.” The disclosure of said U.S. Provisional Patent Application No. 60/918,921 is hereby incorporated herein by reference for all purposes.

### GOVERNMENT RIGHTS IN INVENTION

Work related to the present invention was conducted in the performance of the following contracts: National Science Foundation Contract No. 0618409; National Oceanic and Atmospheric Administration Contract No. SA7528068-1: and National Oceanic and Atmospheric Administration Contract No. SC3527712-E.

### BACKGROUND OF THE INVENTION

1. Field of the Invention

The present invention relates to a transfection system for Perkinsus species, including a transfection vector and methodology useful for transfection of Perkinsus species to study their genomic character and to achieve overexpression of Perkinsus expression products. The invention relates in an additional aspect to proteins of interest for intervention against mollusk diseases, and associated methods.

2. Description of the Related Art

The present invention relates to a transfection system for Perkinsus species, including a transfection vector and methodology useful for transfection of Perkinsus species to study their genomic character and to achieve overexpression of Perkinsus expression products.

The phylum Apicomplexa is constituted exclusively by protozoan parasites. Toxoplasma, Cryptosporidium, and Cyclospora are serious public health concerns since they not only pose a threat to immunosuppressed individuals (e.g. AIDS, transplanted, or cancer patients) and pregnant women (Toxoplasma), but also because sporadic outbreaks can occur, disrupting water and food distribution (Cryptosporidium and Cyclospora). These organisms are also listed by the NIAID as Category B Bioterrorism Agents because infection occurs via oral transmission and the resistant oocyst remain viable for long periods of time in soil and water.

One of the best characterized Apicomplexan parasites is Plasmodium falciparum, the etiological agent of malaria that causes over a million deaths per year, predominantly children. Members of this genus have been exhaustively studied during the last century. However, the lack of an effective vaccine, the rapid appearance of strains resistant to newly developed antimalarial drugs, and the impact of the disease, largely confined to the less favored regions of the world, has made intervention challenging. Livestock is also threatened by Apicomplexan parasites, especially Eimeria and Leucocytozoon in poultry, and Theileria and Neospora in cattle.

One of the main drawbacks for developing vaccines and new drugs has been the difficulty in obtaining large amounts of the necessary immunogens and putative drug targets using heterologous expression systems, due to either a lack of expression or expression as insoluble inclusion bodies.

During the last decade many genomes of the most relevant parasites of humans and livestock have been cloned. With the mining of these genomes and the identification of genes of interest, the need has become evident for heterologous expression systems as fundamental tools for protein function/structure studies, antigen production, screening and profiling of candidate drugs, and understanding their mechanisms of action.

Heterologous systems that have been used with varying degrees of success in Apicomplexa include prokaryotic (Escherichia coli), yeast (Saccharomyces cerevisiae, Pichia pastoris) and other eukaryotic cells (CHO cells, Drosophila cells, the ciliate Tetrahymena thermophila), virallinsect, and wheat cell-free expression systems. Nonetheless, there is not a single expression system universally suitable for all applications, and all systems have serious drawbacks for specific applications.

There is a need in the art for development and validation of a heterologous system for production of Apicomplexa proteins, given that available systems perform sub-optimally and are characterized by lack of protein expression, low protein yield, and inactive/misfolded proteins.

### SUMMARY OF THE INVENTION

The present invention relates to a transfection system for Perkinsus species, including a transfection vector and methodology useful for transfection of Perkinsus species to study their genomic character and to achieve overexpression of Perkinsus expression products. The invention relates in an additional aspect to proteins of interest for intervention against mollusk diseases, and associated methods.

In one aspect, the invention relates to a vector, selected from the group consisting of:
(i) vectors comprising PmMOE;
(ii) vectors having a restriction map as shown in FIG. 1A;
(iii) vectors expressing heterologous DNA encoding a protein targeted to a trophozoite wall of Perkinsozoa;
(iv) vectors transfectionally present in a Perkinsus trophozoite and expressing heterologous DNA encoding one or more Apicomplexa proteins;
(v) vectors comprising pPmMOE;
(vi) vectors comprising pPmMOE-eGFP; and
(vii) vectors expressing heterologous DNA encoding one or more Apicomplexa proteins.

In another aspect, the invention relates to a Perkinsus trophozoite transfected with such a vector.

A further aspect of the invention relates to a heterologous expression system for Apicomplexia genes, comprising a recombinant vector expressing heterologous DNA encoding an Apicomplexa protein.

In another aspect, the invention relates to a heterologous expression system comprising a recombinant vector expressing heterologous DNA encoding a protein targeted to a trophozoite wall of a protozoan parasite.

Another aspect of the invention relates to a Perkinsus host organism transformed for heterologous expression of Apicomplexa proteins.

In a further aspect, the invention relates to a vector for transfecting Perkinsus trophozoites, comprising heterologous DNA encoding a protein targeted to a trophozoite wall of Perkinsozoa.

Another aspect of the invention relates to a composition comprising Perkinsus cell walls carrying Apicomplexa antigens fused to Perkinsus gene products that are targeted to said walls.

Still another aspect of the invention relates to a transfection method for forming a transformant of Perkinsus marinus, comprising transfecting Perkinsus marinus trophozoltes with a pPmMOE-eGFP construct via electroporation.

Other aspects, features and advantages of the present invention will be more fully apparent in the ensuing disclosure and appended claims.

### BRIEF DESCRIPTION OF THE DRAWINGS

FIG. 1 illustrates the genetic manipulation of Perkinsus. FIG. 1A illustrates the pPmMOE-GFP vector. FIG. 1B shows trophozoites transfected with GFP-tagged pPmMOE, a gene unique to the parasite and considered to be targeted to the parasite's cell wall. FIG. 1C shows fluorescence maintained with no drug selection for more than seven months after the transfection.

FIG. 2 is a graph of Optical density (OD₆₀₀) as a function of time, in days, for various selectable marker candidates for their effect on Perkinsus proliferation.

FIG. 3 is a schematic representation of constructs for transfection. The first construct for transfection includes an MOE promoter and the heterologous gene tagged with GFP. Subsequent constructs include secretion sequences (signal/leader) and affinity purification tags.

### DETAILED DESCRIPTION OF THE INVENTION

The present invention relates to a transfection system for Perkinsus species, including a transfection vector and methodology useful for transfection of Perkinsus species, e.g., the protozoan parasite Perkinsus marinus, to study their genomic character and to achieve overexpression of Perkinsus expression products. The invention relates in additional aspects to proteins of interest for intervention against mollusk diseases, and associated methods, and to heterologous expression systems for Apicomplexia genes.

The disclosure of U.S. Pat. No. 6,326,485 issued Dec. 4, 2001 to Vasta, et al. for “Assay for perkinsus in shellfish” is hereby incorporated herein by reference, in its entirety.

Prompted by the progression on the <i>Perkinsus marinus</i> genome sequence and functional genomics, the present inventors have developed a transfection system that is useful for investigating this parasite. ESTs derived from two trophozoite libraries have enabled identification of a highly represented transcript from an intronless gene (PmT) coding for a putative 136 aa protein. A plasmid (pPmT) containing one Kb upstream and downstream of the gene is utilized as the basis for several constructs for transfection of <i>P. marinus</i>. A <i>Pci<li> I restriction site just upstream of the stop codon is employed to fuse EGFP directly to PmT without any linker sequence (PmT-GFP).

For transfection a Cell Line Optimization Nucleofector Kit in the Nucleofeclor™ II (Amaxa) can be used. Using the manufacturer' solutions L and V and 7 programs set-ups, a variety of conditions can be assessed. In one illustrative investigation, a total of 18 conditions was tested. In such testing, viability upon transfection varied between <0.1 and 45%. Fluorescence microscopic analysis of transfected trophozoites revealed that PmT-GFP was highly expressed. The best combination of transfection solution and program resulted in 10% viability and 37.8% efficiency, with fluorescence detected for 17 hours duration.

Using the same methodology and construct <i>P. olseni</i> (=<i>P. atlanticus<li> and other strains of P. marinus were also transfected. The invention represents the first known development of transfection methodology for a member of the Perkinsozoa. The transfection vector can be optimized in any suitable manner, e.g., by identifying the minimum fusion protein to achieve expression, testing other putative gene promoters, secretion signals, selectable markers, and incorporating a polylinker to facilitate the cloning of genes of interest.

The present invention has resulted in the validation of the protozoan Perkinsus marinus as a heterologous system for producing recombinant proteins from Apicomplexan parasites of human or veterinary relevance. Transfection vectors can be designed and constructed, and the transfection conditions can be optimized for expressing recombinant proteins that have proven either difficult to produce in other systems or remain insoluble or inactive.

While there is no universal heterologous system for all proteins of interest, Perkinsus has significant advantages over other expression systems, as a suitable alternative when other available systems fail to perform effectively for Apicomplexa proteins.

The invention in one aspect relates to a vector for transfecting Perkinsus trophozoites. Perkinsus has been found to be sensitive to several drugs that can be used as selectable markers. The transfection conditions can be readily optimized, and the transfection vectors can incorporate selectable markers, multiple cloning sites (MCS), and tags that are useful for both purification and as reporters. The techniques of the invention enable highly expressed constitutive and inducible genes from Perkinsus to be efficiently produced.

The invention in another aspect relates to characterization of Perkinsus subcellular targeting and secretion pathways and mechanisms. Most protozoan parasites use distinct mechanisms for secretion depending on the nature and the targeting of the protein. The invention in another embodiment relates to the development of a heterologous system for expression of proteins involving (a) mining the genome database for subcellular targeting and secretion signals, and (b) characterization of secretion signals and the mechanisms for targeting and secretion.

Currently available heterologous expression systems are non-optimal for production of recombinant proteins from Apicomplexa. To our knowledge, Perkinsus is the only member of the early branch (Perkinsozoa) close to the divergence point leading to the Apicomplexa and oxyrrhis-dinoflagelates for which cell-free, defined culture media have been developed, genomic sequencing has been conducted, and transfection has been achieved. To validate Perkinsus as a heterologous system for the production of recombinant proteins for functional and structural studies, and production of immunogens for vaccine development, representative proteins are selected from well-characterized Apicomplexa, and the corresponding genes are cloned into the expression vector(s), following which protein expression yield and quality are evaluated.

The invention in specific embodiments utilizes the protozoan Perkinsus marinus.

A variety of challenges confront efforts to isolate and purify proteins from Apicomplexa. Inasmuch as the phylum Apicomplexa contains numerous protozoan parasites that cause disease of both medical and veterinary importance, various genomes of Apicomplexan parasites have been sequenced. With the small genome sizes relative to other metazoans and the rapid improvements in annotation algorithms, an enormous amount of genomic information for these organisms is being developed. Unfortunately, genome annotation also underscores the lack of known function for most of the predicted genes.

Although there are numerous novel approaches for predicting function based on pattern of expression and interactive networks, the ultimate proof of function requires individual biochemical and biophysical. characterization, and a functional study of the protein of interest in the context of the cell type or stage where it is expressed. Apicomplexa are no exception, but rather represent more ultimate challenges since most species are obligate intracellular parasites.

These challenges include complex life cycles. The study of most of the Apicomplexa parasites of human relevance has to rely on alternative animal or in vitro models. Fortunately, the combination of human cell lines and particular life cycle parasite stages has been used to significantly advance this field. However, some genes of interest may be expressed in organs for which cell lines are lacking, or, more fundamentally, the method(s) necessary to obtain the parasite in the life stage that infects a particular cell type may not have yet been developed.

These difficulties can be partially overcome by using alternative animal models. For example, Plasmodium berghei is the murine model for P. falciparum). In the case of T. gondii, the model is more manageable since the final host is the domestic cat. For Cryptosporidium spp. infected cows must be maintained year round in qualified facilities with trained personnel, for the purification of cysts for experimental challenges, genomics, and functional studies. Rats have been proposed as an alternative, more manageable animal reservoir.

The challenges related to Apicomplexa also include problems associated with large-scale production. The complexity associated with the life cycles of these parasites, including the fact that most of them are obligate intracellular parasites, poses a challenge to large scale protein production, even when the protein of interest is expressed successfully in the in vitro model.

This problem can be overcome by over-expressing the protein under control of promoters specific for the parasite stage propagated in vitro. Prior to purification of the protein of interest, however, the parasite needs to be isolated from the cell host, with the inherent risk of contamination by the host proteins.

In addition, the production of large amounts of protein necessary for most applications requires a dedicated, complex infrastructure. Although improved technologies have reduced the amount of protein required for large screenings (e.g., crystallization, drug screening), the application of high throughput methodologies still requires high protein concentrations. Usually, the protein yield varies considerably depending on the protein and expression system used.

As a specific example, the average yield of the recombinant merozoite surface protein 1 (MSP-1) in an E. coli host has been reported as 48 mg/l of bacterial culture. In large-scale expression studies on 63 target proteins, yields of from 0.9 to 406.6 mg/l of rich media have been determined. However, the expression of recombinant proteins can also result in amino acid substitution and modification. For example, the hisrich protein from P. falciparum expressed in E. coli has been reported to result in the isolation of a heterogeneous protein.

The challenges related to Apicomplexa also include problems associated with genome bias. Some organisms such as Plasmodium are characterized by an AT rich genome, which makes the production of proteins in E. coli much more difficult. This problem can be partially resolved by using optimized or harmonized plasmid vectors, which can greatly increase levels of over-expressed proteins. One alternative heterologous system for the expression of genes from AT rich genomes is the ciliate Tetrahymena termophHa, which also has an AT rich genome. However, this system has not been extensively used.

Recent studies including 1,000 Plasmodium open reading frames (ORF) and 1,008 genes of Apicomplexa from seven available genomes, indicate that codon usage and the percentage of AT appears not to play a significant role on expression. Larger proteins and proteins with high pI also proved much more difficult to produce and interestingly, no full length apicoplas-targeted proteins produced were soluble, raising additional doubts about the suitability of prokaryotic systems.

The challenges related to Apicomplexa also involve issues associated with post-translational modifications. Native eukaryotic proteins contain various post-translational modifications of amino acid side chains that are required for adequate in vivo biological function. These modifications incorporate an additional level of diversity to protein function by providing ˜200 distinct aa units in addition to the 20 aa given by the genetic code.

Protein modifications in protozoan parasites, such as glycosylation, are particularly important in the context of vector-pathogen dynamics, and have been difficult to study due to the lack of molecular tools; most of the published work has focused on the vector. Within protozoan parasites, the Kinetoplastida (Trypanosoma and Leishmania) are the most studied. Recent evidence indicates that the Plasmodium ookinete has carbohydrate recognition properties.

Other post-translational modifications of amino acids and addition of lipids may be required for production of recombinant proteins that closely resemble the authentic molecular species, and include, without limitation, removal of N-terminal methionine, acylation, phosphorylation, sulfation, γ-carboxylation, 13-hydroxylation, and protein subunit assembly.

The complexity of the life cycle of most Apicomplexa parasites, especially those causing disease in humans and livestock, has limited the large-scale production of proteins of interest. Few crystal structures have been resolved for Apicomplexa proteins, further indicating that heterologous expression and crystallization of Apicomplexan proteins have only had limited success. Recently, a large high-throughput study targeting P. falciparum selected gene orthologs from other Plasmodium species, C. parvum, and T. gondii resulted in 304 (30.2%) purified soluble recombinant proteins, with 36 structures resolved (3.8%) In another recent study, from a total of 1,000 ORFs from P. falciparum tested using an E. coli protein expression system, less than 7% were expressed as soluble proteins.

The method of choice for heterologous expression is in part determined by the objective pursued. Large-scale functional genomic studies, and vaccine and drug development, can be greatly accelerated by the application of high-throughput approaches. Protein expression systems can be assigned to four broad categories: small proteins (<88 aa), expressed as fusion proteins; secreted polypeptides (80-500 aa), preferably expressed in yeast or mammalian cells; very large (>500 aa) secreted and cell surface proteins, usually expressed in mammalian systems; and non-secreted proteins (>80 aa), for which the choice of the system depends on the nature of the protein. Prokaryotic systems, eukaryotic systems, insect cells/viral systems and wheat cell-free systems can be employed as expression systems.

Concerning prokaryotic systems, despite the complexity involved in purifying proteins from inclusion bodies and the presence of endotoxins, the use of the E. coli expression system is the most popular choice for expressing Apicomplexa proteins. Of 303 target single-exon P. falciparum genes selected, only 7% of the genes induced antibody titers against erythrocytic or sporozoite stages. Improvements in such technology include the use of novel strains to increase solubility, or methodologies for solubilizing the inclusion bodies and purifying the protein of interest. Alternatively, immunogenicity can be improved by fusing the parasite antigen to a protein anchor displayed on the cell wall of the heterologous organism to use in oral immunizations. Although optimized plasmids may improve yields of the full-length proteins, the overall success rate for soluble recombinant Apicomplexan proteins in prokaryotic systems remains low.

Concerning eukaryotic systems, such systems have been used to produce Apicomplexa proteins. These include universal systems such as yeast, mammalian, and insect cells, but also systems specifically developed by exploiting phylogenetic affinities. PfCP-2.9 was produced in Pichia pastoris as the secreted form, with a yield of 2.6 mg/L Approximately 1 gm/l of final product was obtained from a three-step purification process. P. pastoris was also used to produce Pvs25 (zygote/ookinet surface), a transmission blocking vaccine candidate. Neospora caninum can express genes of the phylogenetically-related T. gondii. In some instances, the phylogenetic boundaries are not an obstacle; engineered T. gondii is-4 mutants can express the Leishmania antigen KMP-11 and elicit a specific OG immune response in BALB/c mice. Mammalian cells (e.g., COS-7 cells) can be used to express proteins from Toxoplasma and Plasmodium for binding assays and vaccine candidate tests.

Insect cells and viral systems can be used to produce Apicomplexa proteins. Virus-mediated expression is an approach for the rapid production of high levels of heterologous proteins in insect cells. As a transient expression system, the insect systems are capable of providing milligram quantities of the recombinant protein of interest with desirable post-transcriptional modifications. Bombyx mori nuclear polyhedrosis virus-silkworm expression system has been used to produce P. falciparum MSP-1. Viruses constitute one of the most used systems for developing vaccines and have been used as both delivery systems and as adjuvants. These include P. falciparum circumsporozoite protein in a modified hepatitis B virus core particle, recombinant poxvirus FP9, or a modified vaccinia virus Ankara, and the malaria protein B in yellow fever 170 virus. T gondii surface antigens expressed in replication-deficient recombinant adenoviruses induce a protective immune response against infection in mice. MVA ROP2 vaccinia virus recombinant is also a vaccine candidate for toxoplasmosis.

Wheat cell-free expression systems can be used to produce Apicomplexa proteins, e.g., a functional PfDHFR-TS system.

Perkinsus can be used as an alternative system to produce recombinant proteins from Apicomplexa, in accordance with the present invention, in one aspect thereof. Perkinsus marinus is a protozoan parasite responsible for the decline of the oyster populations in the Chesapeake Bay since the 1950s. Perkinsus has been placed in different phylogenetic groups since it was first described. The phylum Perkinsozoa, like the Apicomplexa, includes only parasites. It is considered one of the earliest diverging groups of the lineage leading to dinoflagellates, albeit close to the ancestor from which the ciliates, dinoflagellates, and Apicomplexa originated. Indeed, this phylogentic position at the base of the branch makes the Perkinsozoa a key taxon for understanding adaptations (e.g., parasitism) within the Alveolata and holds the potential for Perkinsus to be more suitable than prokaryotes and other eukaryotes in producing recombinant proteins from Apicomplexa.

The Perkinsus marinus life cycle includes a free-living stage (zoospore) and a non-motile vegetative stage (trophozoite). Once inside an oyster Perkinsus can multiply by schizogony intracellularity in the oyster hemocyte or extracellularity when the hemocyte breaks down. Once it reaches the water, the trophozoite enlarges and goes through zoosporulation to generate hundred of zoospores. Infective stages are filtered by neighboring oysters.

Perkinsus shares multiple features with the Apicomplexa. Perkinsus is a facultative intracellular parasite with both a free-living (zoospore) and non-motile vegetative (trophozoite) stages. Once in the oyster gut lumen, trophozoites are phagocytosed by hemocytes (cells resembling vertebrate macrophages). Interestingly, like numerous Apicomplexa (e.g. Toxoplasma, Plasmodium), the parasite is enclosed within a parasitophorous vacuole where it survives killing by the hemocyte oxidative machinery, and acquires nutrients. Mechanisms to survive the intracellular killing include the use of superoxide dismutases (PmSOD) and ascorbate-dependent peroxidase activity. Interestingly, a similar role has been attributed to Toxoplasma and Plasmodium SOD. Like the erythrocytic stage of Plasmodium, Perkinsus also proliferates by schizogony.

Numerous Apicomplexa life cycle stages (e.g., oocyst in Plasmodium and Cryptosporidium, Perkinsus trophozoites) are also surrounded by a well-developed glycosylated cell wall structure. One of the distinctive structures of Perkinsus, however, is the presence of a large vacuole containing one or more electron-dense inclusions (vacuoplasts) that may be similar to volutin, a complex molecule containing large amounts of orthophosphate polymers, nucleoproteins, and lipids. These structures mostly occur as cytoplasmic granular inclusions (granules) in certain bacteria, yeasts, and protozoa, which serve as intracellular phosphate reserves.

Mature zoospores also contain a large vacuole and organelles that resemble the Apicomplexa apical complex, consisting of an open conoid, rhoptries-like structures, and conoid-associated micronemes. These morphological features resembling the apical complex are inferred to result from external and internal factors, which in the case of Apicomplexa and Perkinsozoa may have determined their condition as parasites. This affinity to the apicomplexan cellular architecture enables Perkinsus more likely to express heterologous proteins than other routinely used systems.

There is indirect evidence for the presence of a plastid in Perkinsus. Apicoplasts are DNA-containing organelles of eukaryotic cells that have evolved via a secondary endosymbiosis. First, engulfment of photosynthetic cyanobacteria took place, and later, heterotrophic protists enslaved the photosynthetic algae. These events gave rise to a group of organisms called the chromalveolates, which includes the Apicomplexa, that contain these secondary plastids.

The structural evidence for the presence of a plastid in Perkinsus has remained elusive, but biochemical evidence supported by molecular data derived from the present inventors' ongoing genomic studies has provided mounting evidence for its presence. In vitro-cultivated parasites contain transcripts of the plant-type ferredoxin and its associated reductase; since this redox pair is exclusively found in cyanobacteria and plastid-harboring organisms, its presence in P. marinus is highly indicative of a plastid.

Similarly, PmSOD2 localizes to distinct cortical patches of unknown function, and this enzyme exhibits N-terminal targeting sequences similar to those characterized in dinoflagellates, suggesting that the structures to which PmSOD2 is targeted may represent a vestigial plastid or plastid-like organelle. Interestingly, in Toxoplasma, TgSOD2 is also targeted to the apicoplast, since this organelle also requires antioxidant protection. Homologs of interest shared by Apicomplexa and Perkinsus can be readily tested for drugs under development or for high throughput drug screens.

Perkinsus can be cultured in cell-free, fully-defined media. Media formulations to culture the parasite trophozoites have been developed. An illustrative example includes the medium including DME:HAMS (1:2) prepared in Crystal Sea Marinemix as the marine salt source, with penicillin G and streptomycin sulfate added, and in which the 5% FBS supplement of the original version is replaced by fetuin. Although maximum growth (doubling time is 16-24 hours) can be achieved under optimized conditions (28° C., 30‰, pH 6.6), the parasite can grow within a relatively wide range of salinity, pH, and temperature.

The trophozoite stage can be grown axenically in large volumes at a density of 108 cells/mL The simplicity of the system and the permissiveness of the parasite make it very easy to use. P. marinus is non-pathogenic to humans and a Biosafety Level 2 fully satisfies the requirements for working with this organism. The parasite can be easily cryopreserved. There are numerous P. marinus isolates and Perkinsus species available from the American Type Culture Collection (http://www.atcc.org) that can be incorporated for comparative purposes in validating Perkinsus as a heterologous expression system to yield the amounts of protein necessary for functional genomics and in vitro drug testing applications.

The P. marinus genome is within the range of other medical and veterinary protozoan parasites (estimated size 70-80 Mb, EI-Sajed, Robledo, and Vasta, unpubl. data). The data is available at http://www.tigr.org/tdb/e2k1/pmg/.

Perkinsus trophozoites can be transfected with high efficiency. Using 5 μg of plasmid DNA, Perkinsus trophozoite transfection efficiency is approximately 40%. The parasite correctly folds GFP, and fluorescence can be detected 14 hours after the transfection, as hereinafter more fully described. The plasmid is maintained despite the lack of a selectable marker, and the cloned transfected cells maintain fluorescence seven months after transfection.

Transfectants can be rapidly cloned, either manually or using fluorescence-activated cell-sorting (FACS). The Perkinsus genome sequence, and transfection methodology of the present invention, enable genetic manipulation of the parasite. In comparison to other unicellular organisms amenable to genetic manipulation that have been adopted as heterologous expression systems (e.g., E. coli, yeast), Perkinsus has been found to afford significant advantage in its phylogenetic affinities with Apicomplexa and an equivalent ease of cultivation and scale-up.

Genomics and Functional Genomics

Initial gene identification studies in P. marinus were carried out in a Lambda DASH genomic DNA library. Preliminary findings suggested that gene density in P. marinus is higher than that of P. falciparum (1 gene per 4 to 5 kb). For example, the genomic region encoding PmNramp, a membrane cation transporter, includes eight exons interrupted by seven short introns (44-61 bp) with canonical splicing signals (GT/AG), features that had already been observed in PmSOD. The 10,298 by contig containing the complete PmNramp locus also carries two partial ORFs (a 26S proteasome regulatory subunit 7 similar to that of P. falciparum, and another having similarity to a folate solute carrier). A Perkinsus expressed sequence tags (EST) effort was initiated, consisting of sequencing clones from two P. marinus Ie ZAP cDNA libraries constructed using either P. marinus propagated in standard culture medium or in standard medium supplemented with C. virginica serum, a factor shown to affect P. marinus growth in vitro.

Two important findings were made in the P. marinus EST database (2,500 sequences): (i) there are many genes whose products are known in other parasite species to be involved in virulence, or are currently being investigated as targets for therapy, and (ii) there are notable different species in the classes of genes expressed either in standard or oyster serum-supplemented cultures. By BLAST analysis of the EST the expression of several candidates for secretion was identified, especially proteases. The deduced amino acid sequence of PmS+EST18 showed a high degree of conservation with serine proteases, and is consistent with extracellular protein character. Secreted or excreted proteases have been postulated as the means for P. marinus to digest and acquire the host nutrients, amino acids and peptides that are necessary for normal cell functions.

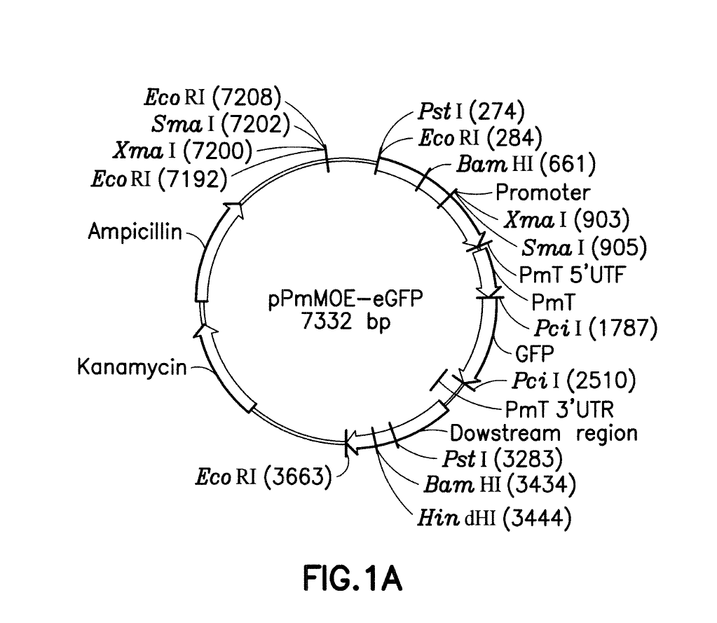

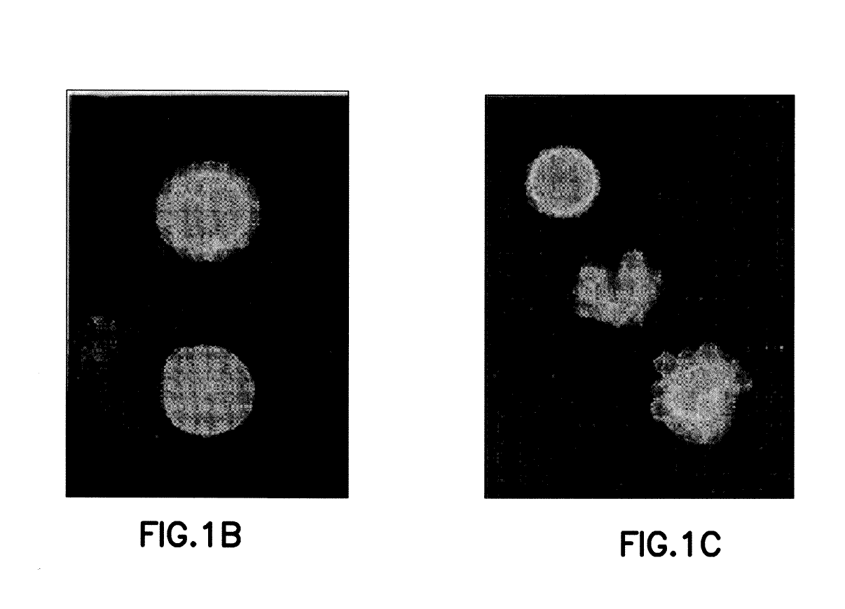

The apparent lack of sexual reproduction in Perkinsus spp. has precluded application of forward and reverse genetic methods to enhance understanding of the parasite's biology. In addition, transfection of P. marinus has been hampered by the lack of suitable vectors and knowledge of sensitivity to most common drugs for selection. The present inventors' EST database analysis and sequencing efforts has enabled the construction of transfection vectors. In assessment of 2,500 ESTs from two cDNA libraries, a highly represented transcript was identified in both libraries that codes for a putative 136 aa protein with no BLAST similarities in GenBank (p<1 0.4). SignalP (0.99) and TargetP (0.93) predicted that this protein is secreted, with a cleavage site at position 26-27 of the predicted protein, highly glycosylated, and apparently targeted to the trophozoite wall. The gene was named MOE (based on the Latin moeniaum, the walls or fortifications of a city). A contig containing the full cDNA and the regions expanding upstream and downstream of the gene was identified in the ongoing genome sequence project and revealed that PmMOE is intronless. An amplicon containing the full gene and expanding 1 kb both upstream and downstream of MOE was cloned into pCR4-TOPO. A Pci I restriction 15 nt upstream of the stop codon was used to ligate eGPF to produce pPmMOE-GFP (FIG. 1A). This vector was used to transfect Perkinsus trophozoites.

FIG. 1 illustrates the genetic manipulation of Perkinsus. FIG. 1A illustrates the pPmMO′E-GFP vector. FIG. 1B shows trophozoites transfected with GFP-tagged pPmMOE, a gene unique to the parasite and considered to be targeted to the parasite's cell wall. FIG. 1C shows fluorescence maintained with no drug selection for more than seven months after the transfection.

Once a gene of interest is selected for any particular parasite, the rigorous demonstration of its function(s) may be challenging. Because the tools for the functional characterization of Perkinsus genes were lacking, the present inventors engaged in the development of transfection methodology. For this, a Cell Line Optimization Nucleofector Kit in the Nucleofector™ II (Amaxa) was used, which together with the optimization kit, provided two electroporation solutions and seven pre-set programs. By using a 5 μg DNA mix of both supercoiled and linearized plasmid to transfect 5×10⁷ trophozoites in the log phase, we were able to detect fluorescent trophozoites using both solutions with all the Nucleofector pre-set programs tested. Survival rate, however, was higher in trophozoites transfected in solution V than trophozoites transfected in solution L. Nevertheless, programs L-029 and D-023 proved to be the most effective for introducing exogenous DNA into Perkinsus trophozoites with both solutions (see Table 1 below).

TABLE 1

| | Solution L | | Solution V | |
|---|---|---|---|---|
| | Viability | Florescence | Viability | Florescence |
| Program | (%) | Score | (%) | Score |
| A-020 | 15.27 | + | 29.80 | + |
| T-020 | 0.51 | + | 0.50 | ++ |
| T-030 | 0.53 | + | 4.00 | ++ |
| X-001 | 0.50 | ++ | 0.50 | ++ |
| X-005 | 0.50 | +++ | 4.76 | +++ |
| L-029 | 0.50 | ++++ | 0.50 | +++ |
| D-023 | 1.50 | +++++ | 0.60 | ++++ |
| No Pulse | 88.12 | None | 78.76 | None |
| T-020 | 4.76 | None | 3.77 | None |

The best combination of survival and fluorescent cells corresponded to electroporation solution V and the program D-023. The efficiency was 37.8%. Fluorescence was detected 14 hours after transfection and after cloning the transfected parasites, fluorescence was still seen seven months after the initial experiment.

Fluorescence microscopic analysis of transfected trophozoites reveled that pPmMOE-GFP was highly expressed and the gene product appeared to be targeted to the parasite wall (FIGS. 1B-1C). This same construct has been used to transfect other Perkinsus strains and species, and to successfully target other Perkinsus proteins under control of MOE promoter. Preliminary evidence indicates that control elements from some Apicomplexa can be recognized by the Perkinsus transcriptional translation machinery to drive gene expression.

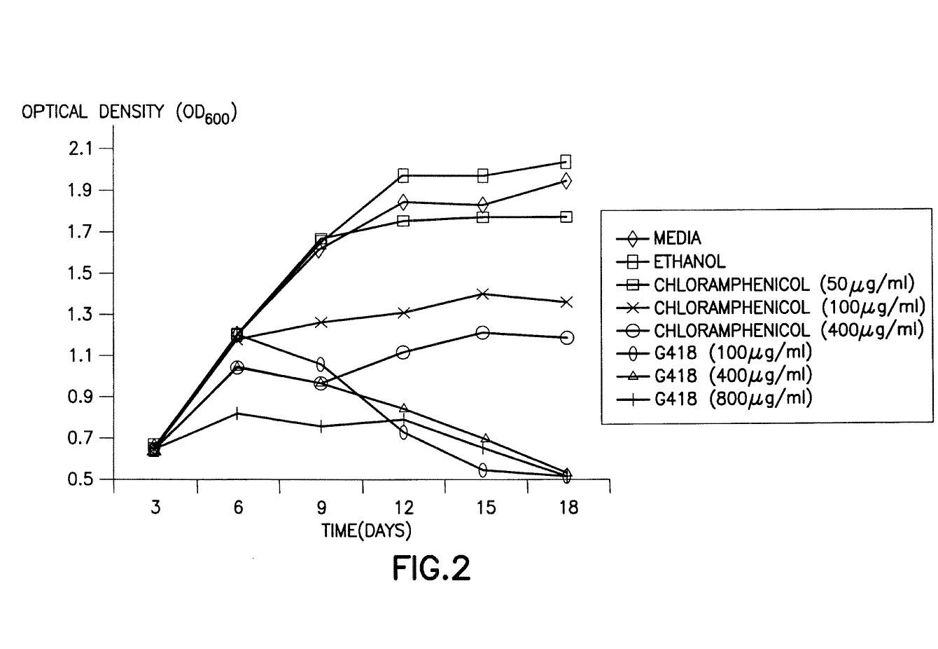

An initial plasmid constructed for transfection lacked a selectable marker, but as indicated above, parasites were still fluorescent seven months after transfection. While not wishing to be bound by hypothesis, the plasmid may be maintained episomally or it may have integrated into the genome. Southern blot analysis indicated that the plasmid had integrated, and peR analysis was consistent with a non-homologous integration. It appears that rearrangement of either vector or target sequences may have occurred. To improve the screening of the transfected cells, it is preferred to incorporate a selectable marker into the transfection vector. Various drugs that have been used successfully as resistance markers in other systems may be employed. Both G418 and chloramphenicol (CAP) inhibited parasite growth (FIG. 2).

FIG. 2 is a graph of Optical density (OD₆₀₀) as a function of time, in days, for various selectable marker candidates for their effect on Perkinsus proliferation. Both CAP and G418 showed inhibition of Perkinsus proliferation, especially G418.

The curves shown in FIG. 2 include, from top to bottom of the graph, at 15 days, the following marker curves: top curve=ethanol; second highest curve=media; third highest curve=chloramphenicol, 50 μg/ml; fourth highest curve=chloramphenicol, 100 μg/ml; fifth highest curve=chloramphenicol, 400 μg/ml; sixth highest curve=G418 (800 μg/ml); seventh highest curve=G418 (400 μg/ml); and lowest curve=G418 (100 μg/ml).

G418 (800 μg/ml) was highly toxic, with visible effects six days after treatment. Lower doses tested (100 and 400 μg/ml) still inhibited parasite growth, with cells dying 15 days after treatment. Pyrimethamine, a drug that interferes with the folic acid pathway, inhibited P. marinus proliferation at 2 mg/ml, and a gene of the folic acid pathway was identified (P. marinus DHFR). Visual inspection of the treated cells indicated changes in the morphology and cell death. These changes were more evident for G418 and pyrimethamine than with the CAP treatment.

Empirical Determinations

Empirical efforts were based on the hypothesis that P. marinus has targeting signals and secretion mechanisms suitable for production of recombinant proteins from Apicomplexa, and that P. marinus can be used as a heterologous system for the expression of Apicomplexa genes. Most protozoan parasites utilize distinct mechanisms for secretion, depending on the nature of the protein and its targeted organelle. Similarly, several P. marinus proteins can be detected in a particular organelle and in the spent medium.

Preliminary results indicated that 5 μg of pPmMOE-GFP can be used for transfection with a 40% efficiency (5×107 trophozoites in the log phase) even though most Apicomplexa organisms use 20-50 μg of linearized plasmid. Using solution V, additional programs derived from D-023 and L-029 can be utilized for improving transfection efficiency, involving varying the cell numbers, and using cells at different points in the growth curve (lag, log, and stationary). After optimization of cell numbers, the plasmid concentration can be titrated by using variable amounts of plasmid DNA (e.g., 5, 10, 20, and 50 μg). Transfection efficiency can then be compared between circular and linearized plasmid. The vector and the optimized transfection conditions are used in the next following step.

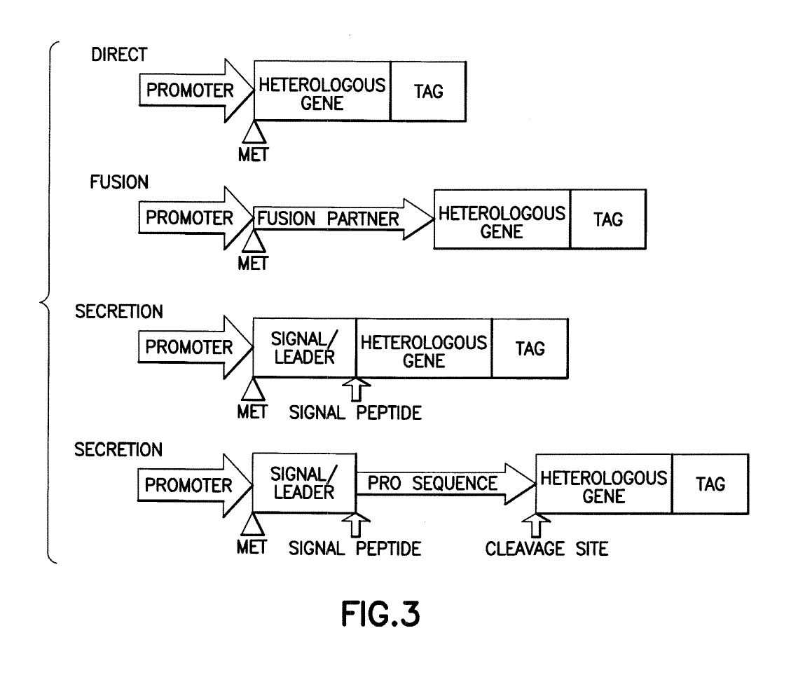

In this step, selectable Plasmodium markers, MCS, purification tags, and fluorescent reporters are incorporated in the transfection vector: To accelerate cloning, screening, and purification of the recombinant proteins, required features can be incorporated into the plasmid. A Cryptosporidium selectable marker can be incorporated to facilitate cloning of transfectants. Preliminary data indicate that Perkinsus trophozoites are sensitive to a series of drugs used as selectable markers. G418, an aminoglycoside, similar in structure to neomycin, gentamycin, and kanamycin, blocks protein synthesis in eukaryotic cells by interfering with ribosomal function. Expression of the bacterial APH gene in eukaryotic cells therefore results in detoxification of G418. Expression of the DHFR-TS gene results in detoxification of the drug pyrimethamine, to which Perkinsus is also sensitive. Chloramphenicol acetyl transferase driven by the MOE promoter can be used to detoxify CAP. To facilitate gene cloning, a MCS can be incorporated. Fusion tags (e.g., His-, S-, c-myc-tag), preceded by a cleavage site, can be used to facilitate purification. In addition to GFP, additional plasmids with other fluorescent reporters (yFP, RFP, CYP) can be constructed. If necessary, for facilitating mRNA processing, intervening sequences can be incorporated into the plasmid (FIG. 3).

FIG. 3 is a schematic representation of constructs for transfection. The first construct for transfection includes an MOE promoter and the heterologous gene tagged with GFP. Subsequent constructs include secretion sequences (signal/leader) and affinity purification tags.

To provide for integration of pPmMOE-GFP into the genome, a vector specifically designed for integration can be provided. Constitutively and inducible highly expressed genes can be identified, and MOE or alternative constitutive and inducible promoters can be employed.

EST data can be generated from a non-normalized library, to identify a group of ESTs that are highly represented, as in Table 2 below.

TABLE 2
Summary of the Most Represented ESTs of P. marinus

| ESTcluster | n | E value | Protein function | Closer hit |
|---|---|---|---|---|
| CL1_C14 | 190 | 3 × e⁻⁵⁸ | Surface protein D | Theileria |
| CL5_C1 | 188 | No hit | Unknown | — |
| CL6_C3 | 157 | No hit | Unknown | — |
| CL11_C1 | 140 | 1 × e⁻⁶⁵ | Cystein endopeptid. | Arabidopsis |
| CL3_C3 | 140 | 3 × e⁻⁸ | Angiogenic factor | Cryptosporidium |
| CL9_C1 | 127 | 0.002 | Leishmanolysin | Tetrahymena |
| CL13_C1 | 113 | 0.0 | Peptide exporter | Ostreococcus |
| CL2_C9 | 113 | No hit | Unknown | — |
| CL7_C4 | 103 | 2 × e⁻¹¹⁶ | Fructose-biP aldol. | Dictyostelium |
| CL2_C8 | 102 | 6 × e⁻⁵ | Pig377 | Plasmodium |
| CL11_C1 | 100 | 3 × e⁻⁵⁰ | 60S rib. prol. L23 | Cryptosporidium |
| CL15_C1 | 94 | No hit | Unknown | — |
| CL14_C2 | 93 | No hit | Unknown | — |
| CL16_C1 | 93 | 2 × e⁻¹²⁷ | ABC transporter | Macaca |
| CL10_C1 | 92 | 0.18 | Scarp-like | Microscilla |
| CL51_C2 | 90 | 0.0 | HSP 70 | Crypthecodinium |

The EST can then be mapped to the genome, e.g., with incorporation of an additional 1 kb both upstream and downstream of the selected gene into the transfection vector. In addition, the Perkinsus genome can be screened for more highly-expressed genes, and for tightly regulated genes. To perform these genomic screens, microarrays and promoter traps can be used.

Microarrays enable simultaneous monitoring of the expression of thousands of genes and, unlike conventional Northern blot and PCR-based techniques, have the potential to rapidly identify highly expressed genes. ESTs can be determined for unigenes, followed by assignment of the ESTs to the genome, and annotation to establish a full gene catalogue for the Perkinsus gene index. Based on this gene index, long oligonucleotide (e.g., −60 mers) can be obtained commercially, and spotted directly onto the microarray. The main advantages of using oligonucleotides are that they can be designed to guarantee appropriate thermodynamic properties for hybridization, thereby ensuring that no cross-hybridization occurs. Target search and retrieval, domain target identification, and array annotation are then carried out. Probe design next can be conducted to ensure target-specific probes for target sequences. For the array layout, randomized probe placement on the microarray, defined probe placement, and experimentation with several formats (e.g., between −11 K and −44K spots per slide) can be carried out to assess induction of highly expressed genes (tightly regulated promoters) in response to abiotic factors (such as temperature, salinity, and free radicals) using a fully-defined medium to facilitate the purification of the recombinant proteins. Gene up-regulation can then be verified by Northern blot and RT-PCR. The full cDNA can be amplified using RACE (SMART, Clontech, or GeneRacer, Invitrogen). cDNA then can be mapped to the genome, and the upstream region including the gene UTR incorporated into the transfection vectors.

Genomic screens can be carried out using promoter traps. P. marinus contains a remarkable number of intracellular compartments, including some that may be unique to the Perkinsozoa. A GFP-based motif-trap can be used to identify strong promoters for heterologous expression, as well as to identify subcellular localization of candidate proteins, and subsequently, the signals responsible for sorting. Promoter trap is a technique usefully applied to protozoan parasites. This technique requires the integration of a linearized GFP construct lacking promoter and initiator methionine codon. Fluorescent parasites, indicative of GFP fused in-frame into a coding region, are sorted by FACS followed by screening by fluorescence microscopy for those containing GFP targeted to specific intracellular compartments. Application of this technique requires the design of vectors for integration into regions encoding exons, and vectors with a splice acceptor site for integration into regions encoding introns. As sequences for integration, P. marinus RNA genes (est. on the order of 560 copies per genome) or transposase sequences (e.g., a HAT-like transposase in the Perkinsus genome) can be used. Integration can then be characterized.

Characterization of Perkinsus subcellular targeting and secretion pathways and mechanisms can be characterized in several ways, including genomic mining and matrix-assisted laser desorption/ionization-time of flight-mass spectrometry (MALDI-TOF-MS).

In genomic mining, signal peptides (or signal anchors) and transmembrane domains can be predicted using SignalP 3.0 and TargetP 1.1. SignalP 3.0 uses an algorithm that predicts the presence and location of signal peptide cleavage sites in amino acid sequences from different organisms (see http://www.cbs.dtu.dk/services/SignalP/). TargetP 1.1 predicts the subcellular location of eukaryotic proteins, and is based on the predicted presence of any of the N-terminal presequences: chloroplast transit peptide (cTP), mitochondrial targeting peptide (mTP), or secretory pathway signal peptide (SP) (see http://www.cbs.dtu.dk/services/TargetP/).

MEME (Multiple Expectation-maximization for Motif Elicitation) can subsequently be employed. MEME is a software tool that is used to discover motifs shared by a set of protein sequences in a fully automated manner, and the Motif Alignment and Search Tool (MAST), a tool for searching biological sequence databases for sequences that contain one or more of a group of known motifs, can also be employed. Both programs are usefully applied to intracellular parasites. Predicted signals can be incorporated into the plasmid under control of MOE to verify secretion (Western blot) or subcellular localization (confocal laser scanning microscopy). Signals can then be characterized for subcellular localization since some of the recombinant proteins may need to be targeted to a particular organelle to retain function.

Matrix-assisted laser desorption/ionization-time of flight-mass spectrometry (MALDI-TOF-MS) is also usefully employed to identify secretion signals Similar to other intracellular parasites, after having been engulfed by the oyster hemocytes, Perkinsus trophozoites reside within a parasitophorous vacuole, which forms an interface between the host cell cytosol and the parasite surface. It is likely that the vacuole protects the parasite from potentially harmful substances, but allows its access to essential nutrients. In malaria, the vacuole acts as a transit compartment for parasite proteins in route to the host cell cytoplasm.

Secreted or excreted proteases, e.g., used by P. marinus to obtain from the oyster the metabolic substrates required for normal cell functions and proliferation, can be identified in the culture medium by use of two-dimensional gel electrophoresis (2DE) and MALDI-TOF-MS analysis. These proteins can be grouped to identify conserved leader sequence motifs for protein sorting. P. marinus can be grown in fully-defined medium, and cells can be harvested by centrifugation (e.g., at 480 g for 10 min) Supernatants can be concentrated and 2DE run, with spots on 2DE gels analyzed using suitable software, e.g., ImageMaster Software v4.01 (Amersham Pharmacia Biotech), with attention to spots that are indicative of highly expressed secreted proteins. Tryptic-digested proteins can be identified by comparing peptide mass fingerprinting data from MALDI-TOF spectrometry with DNA or protein databases at NCBI using ‘Mascot in home’ software (see www.matrixscience.com). Liquid chromatography/electrospray ionization-tandem mass spectrometry (MUDPIT) can also be used as an alternative to MALDI-TOF-MS. A repertoire of secreted proteins can then be identified, to determine signal sequences that can be incorporated into transfection vectors.

Once the genes of interest have been identified, the secretion mechanisms can be characterized. Signal sequences identified by genome mining (SignalP 3.0, TargetP 1.1, MEME, MAST) and MALDI-TOF-MS can be manually cured. Representatives of the remaining proteins can then be fused to the N-terminus of GFP under MOE or other validated promoters to verify secretion, and confocal laser scanning microscopy can be employed to characterize the secretion pathway.

In order to carry out expression in Perkinsus of selected Apicomplexa genes, representative proteins from well-characterized Apicomplexa species are selected, focusing on (a) parasites of human and veterinary relevance, (b) well characterized gene products, for which no recombinant protein is available, in order to take advantage of the availability of reagents, such as cDNA and antibodies, and (c) unique genes that are shared by Perkinsozoa and Apicomplexa. Representatives that can be tested in Perkinsus from most genera include the recombinant protein targets in Apicomplexa that are tabulated in Table 3 below.

TABLE 3
Examples of recombinant protein targets in Apicomplexa

| Organism | Disease | Vaccine candidates |
|---|---|---|
| Plasmodium | Malaria | MSP1(33), MSP1(42), CSP, ABRA, Pls25, LSA-1, AMA-1 |
| Toxoplasma | Toxoplasmosis | SAG1, SAG2, ROP2 |
| Cryptosporidium | Cryptosporidiosis | P23, CP15 |
| Theileria | East Coast fever | p87 |
| Neospora | | NcSAG1, NcSRS2, NcMIC3, NcGRA7, NcsHSP33 |
| Babesia | Babebiosis | Bd37, 12D3 11C5 |
| Eimeris | | EtMIC2 |

Cloning of Apicomplexa ORFs into Perkinsus expression vectors can then be carried out, involving expression, purification, and assessment of the recombinant gene products. Apicomplexa ORFs can be expressed under a MOE promoter or other validated promoter fused to the GFP with an affinity tag. Once a particular ORF is transcribed and translated, the culture can be expanded, followed by performance of Northern and Western blots to determine existence of possible chimeras, product fragmentation, premature termination of translation, and internal starts within the coding region. Recombinant proteins can be purified through lysis (except for secreted proteins), affinity chromatography (e.g., for His-tags, BD Talon Metal Affinity Resins, BD Biosciences), gel filtration (GE Healthcare), and concentration steps (for example, using Amicon spin concentrators from Millipore). Biological activity of recombinant proteins can then be assessed. For example, the mature protease of P. falciparum falcipain-2, a hemoglobinase that plays an important role in the parasite life cycle, can be purified and the biochemical properties characteristic of the recombinant protein, including optimal pH, reducing requirements, and substrate specificity, can be compared to those of falcipain-2 produced in E. coli or other expression system. As another specific example, the activity of recombinant enzyme PfDHFR-TS expressed in Perkinsus can be compared with the active form produced using a wheat cell-free system.

The Perkinsus expression system can be used to produce recombinant proteins for vaccine development. For those recombinant proteins to be used for vaccine development, proper folding can be used as indicative of conserved antigenicity. Recombinant proteins selected for this purpose can be tested by their hydrodynamic behavior, or by the binding of conformation-dependent monoclonal antibodies. For some proteins (e.g., Duffy binding protein), in vitro binding assays can be used for functional comparative studies of the recombinant proteins. Because 3D structures can accelerate advances in vaccine development and drug discovery, selected recombinant proteins produced with Perkinsus, particularly those of large size and high pI, can be subjected to crystallization studies and other characterization and therapeutic studies, to identify protein agents having efficacy for therapeutic applications.

In the use of Perkinsus expression systems, the expressed heterologous proteins produced thereby may have therapeutic efficacy, but undesirable toxicity character. To resolve issues of the toxicity of the gene product, it may be advantageous to clone the proteins under the control of a tightly regulated inducible promoter. The transfected cells in such case will be grown to a late stage in the log phase before inducing the expression of the protein of interest. Cells then will be immediately harvested for protein purification. The same approach can be applied to avoid proteolytic degradation of secreted recombinant proteins. In particular, the protein can be targeted to a compartment where it would not be accessible to proteolytic enzymes. As a further variant, bioprocessors can be incorporated when protein production levels are below desired levels. In the implementation of the heterologous system of the invention for the production of recombinant Apicomplexan proteins, particular protein production may require individual optimization, as in other heterologous systems.

The present invention provides a Perkinsus heterologous expression system that is useful to produce recombinant proteins, e.g., from related parasites of medical and veterinary relevance. This expression system increases the number of genes that can be targeted for protein structure/function studies, production of antigens, and in vitro drug screening. Polymorphisms in pathogen antigens present a complex challenge for vaccine design generally. The heterologous expression system of the invention provides flexibility for easy cloning and recombinant protein production.

The invention also has application in the production and use of highly purified subunit vaccines that require potent adjuvants in order to elicit optimal immune responses: Perkinsus is characterized by the presence of a large wall, which appears to be heavily glycosylated. In one implementation of the present invention, Perkinsus cell walls carrying Apicomplexa antigens fused to Perkinsus gene products that are targeted to the wall (e.g., PmMOE) can work as an inherent adjuvant. Other parasites outside the Apicomplexa group, including Kinetoplastida (e.g., Trypanosoma and Leishmania) and Diplomatida (e.g., Giardia and Trychomonas), which are also relevant human pathogens, could also benefit from this expression system.

In use, the Perkinsus marinus trophozoltes can be transfected with the pPmMOE-eGFP construct by electroporation (e.g., with a Nucleoefector™ II apparatus, Amaxa Biosystems). Using 5×10⁷ Perkinsus and 5 μg plasmid, transfection efficiencies on the order of 37% have been demonstrated. Perkinsus olseni (=P. atlanticus) can be successfully transfected with the same vector, under similar conditions. Other selectable markers can be incorporated in the transfection vector.

### INDUSTRIAL APPLICABILITY

The present invention provides a Perkinsus heterologous expression system that is useful to produce recombinant proteins, e.g., from related parasites of medical and veterinary relevance. This expression system increases the number of genes that can be targeted for protein structure/function studies, production of antigens, and in vitro drug screening. Polymorphisms in pathogen antigens present a complex challenge for vaccine design generally. The heterologous expression system of the invention provides flexibility for easy cloning and recombinant protein production.

1. A vector, selected from the group consisting of:
(i) vectors comprising PmMOE;
(ii) vectors having a restriction map as shown in FIG. 1A;
(iii) vectors expressing heterologous DNA encoding a protein targeted to a trophozoite wall of Perkinsozoa;
(iv) vectors transfectionally present in a Perkinsus trophozoite and expressing heterologous DNA encoding one or more Apicomplexa proteins;
(v) vectors comprising pPmMOE;
(vi) vectors comprising pPmMOE-eGFP; and
(vii) vectors expressing heterologous DNA encoding one or more Apicomplexa proteins.

2. (canceled)

3. The vector of claim 1, including a detection tag comprising GFP.

4. (canceled)

5. The vector of claim 1, wherein the vector expresses expressing heterologous DNA encoding a protein targeted to a trophozoite wall of Perkinsozoa, and wherein said Perkinsozoa comprises Perkinsus marinus.

6-9. (canceled)

10. A Perkinsus trophozoite transfected with a vector according to claim 1.

11. A heterologous expression system for Apicomplexia genes, comprising a Perkinsus host organism and a recombinant vector expressing heterologous DNA encoding an Apicomplexa protein.

12. The heterologous expression system of claim 11, wherein the recombinant vector comprises PmT.

13. A heterologous expression system comprising a recombinant vector expressing heterologous DNA encoding a protein targeted to a trophozoite wall of a protozoan parasite.

14. The heterologous expression system of claim 13, wherein the protozoan parasite is selected from the group consisting of P. olseni, P. atlanticus and P. marinus.

15. A heterologous expression system comprising a Perkinsus marinus transformant expressing a recombinant protein of an Apicomplexan parasite.

16. The heterologous expression system of claim 15, wherein the Apicomplexan parasite comprises a human parasite.

17. The heterologous expression system of claim 15, wherein the Apicomplexan parasite comprises a non-human animal parasite.

18. A Perkinsus host organism transformed for heterologous expression of Apicomplexa proteins.

19. A vector for transfecting Perkinsus trophozoites, comprising heterologous DNA encoding a protein targeted to a trophozoite wall of Perkinsozoa.

20. The vector of claim 19, wherein said Perkinsozoa comprises Perkinsus marinus.

21. The vector of claim 20, wherein said Perkinsus marinus comprises a strain selected from P. olseni and P. atlanticus and P. marinus.

22. A composition comprising Perkinsus cell walls carrying Apicomplexa antigens fused to Perkinsus gene products wherein the gene products are targeted to said walls.

23. The composition of claim 22, comprising PmMOE.

24. A transfection method for forming a transformant of Perkinsus marinus, comprising transfecting Perkinsus marinus trophozoltes with a pPmMOE-eGFP construct via electroporation.
